(19) United States
(12) Patent Application Publication
(10) Pub. No.: US 2025/0082640 A1
(43) Pub. Date: Mar. 13, 2025

(54) Therapeutics for Hyponatremia and Polycystic Kidney Disease

(71) Applicant: University of Cincinnati, Cincinnati, OH (US)

(72) Inventor: Hassane Amlal, West Chester, OH (US)

(73) Assignee: University of Cincinnati, Cincinnati, OH (US)

(21) Appl. No.: 18/287,897

(22) PCT Filed: Apr. 22, 2022
(86) PCT No.: PCT/US2022/025960

§ 371 Date: Oct. 23, 2023

Related U.S. Application Data
(60) Provisional application No. 63/274,489, filed on Nov. 1, 2021.
(60) Provisional application No. 63/178,800, filed on Apr. 23, 2021.

(51) Int. Cl. A61K 31/52; A61K 45/06

(52) U.S. Cl. CPC A61K 31/52; A61K 45/06

(57) ABSTRACT

A novel method for treating hyponatremia or polycystic kidney disease in a mammal is disclosed. The method involves administering to the mammal in need of such treatment a therapeutically effective amount of an adenine analog selected from the group consisting of: 2-chloro-6-aminopurine, its tautomer, racemate, optical isomer and/or pharmaceutically or nutritionally acceptable salt thereof, and a pharmaceutical carrier, 6-(dimethylamino) purine, its tautomer, racemate, optical isomer and/or pharmaceutically or nutritionally acceptable salt thereof, and a pharmaceutical carrier; and combinations thereof.

### CROSS REFERENCE TO RELATED APPLICATIONS

This application is a continuation of PCT Application No. PCT/US22/25960 filed Apr. 22, 2022, which claims priority to U.S. Provisional Application Ser. No. 63/178,800, filed Apr. 23, 2021, and U.S. Provisional Application Ser. No. 63/274,489, filed Nov. 1, 2021, which applications are hereby incorporated by reference in their entirety.

### TECHNICAL FIELD

The present invention relates generally to the field of therapeutics for hyponatremia.

### BACKGROUND OF THE INVENTION

Hyponatremia is a frequent and common electrolyte abnormality, characterized by a decrease in sodium concentration in the blood. The severity of hyponatremia is classified based on the serum sodium levels as mild (131-134 mEq/L), moderate (125-130 mEq/L) or severe (<125 mEq/L). Hyponatremia is developed in conditions associated with impaired water homeostasis, and results from excessive water retention in the kidney in response to increased circulating levels of vasopressin (AVP) or antidiuretic hormone (ADH). Hyponatremia is developed in several clinical conditions associated with the increase in the blood levels of vasopressin. These conditions include congestive heart failure, liver cirrhosis, the syndrome of inappropriate secretion of ADH and cancer. Hyponatremia is a hypo-osmolar state which shifts water movement into brain cells, leading to cerebral edema which progresses to neurological severe disorders. If not treated correctly, hyponatremia leads to morbidity and mortality through seizures, increased intracranial pressure and/or herniation, coma, respiratory arrest, and permanent brain damage. In the United States, the prevalence of hyponatremia spans 3.5 to 6 million patients annually, and direct cost associated with the treatment of hyponatremia is ˜3.5 billion in the United States alone.

Polycystic kidney disease (PKD) is another condition that is highly linked to the activity of vasopressin signaling pathway in the kidney. Autosomal recessive (ARPKD) or dominant (ADPKD) polycystic kidney disease are inherited diseases, which result from mutations in PKDH1 gene for ARPKD or mutations in either PKD1 or PKD2 genes in ADPKD. Both diseases are associated with cyst formation, abnormal differentiation of the tubular epithelium, increased cell proliferation, and increased intracellular levels of cyclic adenosine 3′,5′-monophosphate (cAMP), primarily in renal tubules including the collecting duct system. Cyst growth and progression causes kidney enlargement and destruction of renal parenchyma, ultimately leading to renal insufficiency and end-stage renal disease. Urinary concentrating defect is the cause of vasopressin stimulation during the early stage of PKD, before the onset of renal function decline. ARPKD affects 1 in 20,000 live births and is an important cause of mortality and morbidity in neonates and infants. ADPKD affects 13 million people world-wide and accounts for 5 to 10% of patients with end stage renal disease.

The increased circulating levels of vasopressin stimulates cAMP production via its V2 receptor and leads to increased water reabsorption in the kidney collecting duct through the activation of water channel aquaporin-2 (AQP2), which ultimately leads to dilutional hyponatremia. Vasopressin-induced cAMP signaling through its V2 receptor is also responsible for increased fluid accumulation in kidney cysts and thus contributes to accelerated cyst growth and enlargement in polycystic kidney disease.

Because of the role of vasopressin signaling in the etiology of hyponatremia and in cyst growth and progression in PKD, extensive studies are now focused on targeting V2 receptor using tolvaptan and its derivatives for the treatment and management of these diseases. However, the use of vaptans was shown to be associated with liver injury in ADPKD clinical trials. This observation was confirmed in subsequent in vitro studies showing that tolvaptan causes mitochondrial-induced apoptosis and oxidative stress in primary human hepatocytes. Similarly, experimental animal studies showed that 45 strains of mice treated with a single oral dose of tolvaptan exhibited signs of mitochondrial dysfunction, increased oxidative stress, and activation of immune response and in some strains, changes in bile acid homeostasis contributes the development of liver injury. Overall, vaptans are the first lane of therapeutics with some benefit in the treatment of hyponatremia and management of ADPKD, but physicians and administrators should still pay careful attention to risks of liver injury, cost-effectiveness, and long-term disease outcomes. Hence, there is still an urgent need for the discovery of new molecules, which alter vasopressin signaling mechanism and inhibit fluid transport in the kidney and can be used for better management of hyponatremia and progression of PKD.

### SUMMARY OF THE INVENTION

In one embodiment, the present invention addresses that need with a novel method for treating hyponatremia in a mammal. The method involves administering to the mammal in need of such treatment a therapeutically effective amount of an adenine analog selected from the group consisting of: 2-chloro-6-aminopurine, its tautomer, racemate, optical isomer and/or pharmaceutically or nutritionally acceptable salt thereof, and a pharmaceutical carrier; 6-(dimethylamino) purine, its tautomer, racemate, optical isomer and/or pharmaceutically or nutritionally acceptable salt thereof, and a pharmaceutical carrier; and combinations thereof.

In one embodiment, the mammal is administered 2-chloro-6-aminopurine, its tautomer, racemate, optical isomer and/or pharmaceutically or nutritionally acceptable salt thereof, and a pharmaceutical carrier. In another embodiment, the mammal is administered 6-(dimethylamino) purine, its tautomer, racemate, optical isomer and/or pharmaceutically or nutritionally acceptable salt thereof, and a pharmaceutical carrier. In one embodiment, the mammal is administered a dosage of adenine analog from about 0.075% in food (weight/weight) to about 0.25% in food (weight/weight). In another embodiment, the mammal is administered one or more additional drugs to treat hyponatremia. In one embodiment, the additional drugs are selected from the group consisting of furosemide and vaptans.

In another embodiment, the present invention addresses that need with a novel method for treating polycystic kidney disease in a mammal. The method involves administering to the mammal in need of such treatment a therapeutically effective amount of an adenine analog selected from the group consisting of: 2-chloro-6-aminopurine, its tautomer, racemate, optical isomer and/or pharmaceutically or nutritionally acceptable salt thereof, and a pharmaceutical carrier; 6-(dimethylamino) purine, its tautomer, racemate, optical isomer and/or pharmaceutically or nutritionally acceptable salt thereof, and a pharmaceutical carrier; and combinations thereof.

In one embodiment, the mammal is administered 2-chloro-6-aminopurine, its tautomer, racemate, optical isomer and/or pharmaceutically or nutritionally acceptable salt thereof, and a pharmaceutical carrier. In another embodiment, the mammal is administered 6-(dimethylamino) purine, its tautomer, racemate, optical isomer and/or pharmaceutically or nutritionally acceptable salt thereof, and a pharmaceutical carrier. In one embodiment, the mammal is administered a dosage of adenine analog from about 0.075% in food (weight/weight) to about 0.25% in food (weight/weight). In another embodiment, the mammal is administered one or more additional drugs to treat polycystic kidney disease. In one embodiment, the additional drugs are selected from the group consisting of a lower dose of furosemide and a lower dose of a vaptan. In another embodiment, the adenine analog is administered orally. In one embodiment, the adenine analog is administered via subcutaneous injection. In another embodiment, the adenine analog is administered intravenously.

### BRIEF DESCRIPTION OF THE DRAWINGS

The foregoing summary, as well as the following detailed description of preferred embodiments of the application, will be better understood when read in conjunction with the appended drawings.

FIGS. 1A-1D are a series of graphs showing time-course and dose-dependent effect of R2-ADN on water intake as compared to control rats or to baseline levels of the same treated rats. N=4 rats in each group. FIG. 1A shows the results for the control group. FIG. 1B shows the results for the group given a 0.075% dosage. FIG. 1C shows the results for the group given a 0.15% dosage. FIG. 1D shows the results for the group given a 0.25% dosage.

FIGS. 2A-2D are a series of graphs showing time-course and dose-dependent effect of R2-ADN on urine volume as compared to control rats or to baseline levels of the same treated rats. N=4 rats in each group. FIG. 2A shows the results for the control group. FIG. 2B shows the results for the group given a 0.075% dosage. FIG. 2C shows the results for the group given a 0.15% dosage. FIG. 2D shows the results for the group given a 0.25% dosage.

FIGS. 3A-3D are a series of graphs showing time-course and dose-dependent effect of R2-ADN on urine osmolality as compared to control rats or to baseline levels of the same treated rats. N=4 rats in each group. FIG. 3A shows the results for the control group. FIG. 3B shows the results for the group given a 0.075% dosage. FIG. 3C shows the results for the group given a 0.15% dosage. FIG. 3D shows the results for the group given a 0.25% dosage.

FIGS. 4A-D are a series of graphs showing time-course and dose-dependent effect of R2-ADN on food intake as compared to control rats or to baseline levels of the same treated rats. N=4 rats in each group. FIG. 4A shows the results for the control group. FIG. 4B shows the results for the group given a 0.075% dosage. FIG. 4C shows the results for the group given a 0.15% dosage. FIG. 4D shows the results for the group given a 0.25% dosage.

FIGS. 5A-D are a series of graphs showing time-course and dose-dependent effect of R2-ADN on body weight as compared to control rats or to baseline levels of the same treated rats. N=4 rats in each group. FIG. 5A shows the results for the control group. FIG. 5B shows the results for the group given a 0.075% dosage. FIG. 5C shows the results for the group given a 0.15% dosage. FIG. 5D shows the results for the group given a 0.25% dosage.

FIG. 6A is a graph showing the time-course effects of adenine (0.15%) vs. R2-ADN (0.15%) on urine volume. N=4 to 5 rats in each group. * P<0.05 or ¶P<0.02 vs. baseline or Control.

FIG. 6B is a graph showing the time-course effects of adenine (0.15%) vs. R2-ADN (0.15%) on urine osmolality. N=4 to 5 rats in each group. * P<0.05 or ¶P<0.02 vs. baseline or Control.

FIG. 7A is a graph showing the food intake of rats after being administered R4-ADN [(6-(Dimethylamino) purine]

FIG. 7B is a graph showing the water intake of rats after being administered R4-ADN [(6-(Dimethylamino) purine].

FIG. 7C is a graph showing the urine volume of rats after being administered R4-ADN [(6-(Dimethylamino) purine].

FIG. 7D is a graph showing the urine osmolality of rats after being administered R4-ADN [(6-(Dimethylamino) purine].

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Adenine, a natural nucleobase molecule, impairs renal function, food behavior, and fluid balance in rats. Adenine at high dose causes a significant fluid loss by decreasing the gene expression and protein abundance of both water channel aquaporin 2 (AQP2) in the collecting duct system and the salt transporter NKCC2 in the medullary thick ascending limb of rat kidney. The present invention identified adenine analogs with a modified molecular structure, which can mimic the effects of adenine on renal handling of salt and water in rat.

The present invention uses lower doses of adenine analogs to treat hyponatremia and limit the progression of polycystic kidney disease without any major secondary effect on the body. In addition, adenine analogs are less expensive than vaptans. Therefore, all patients can have access to their use for better management of hyponatremia and/or polycystic kidney disease.

The adenine analogs of the present invention can be taken orally at lower doses. In one embodiment, “lower doses” means from 0.075% to 0.15% added to food (weight/weight). They are slow acting molecules that increase water loss by the kidney over time. This should prevent a rapid correction or overcorrection of sodium levels in the blood of hyponatremic patients. Also, if needed, the adenine analogs of the present invention can be used in combination with lower doses of other drugs to inhibit cyst fluid filling and cyst growth in polycystic kidney disease. Examples of other drugs include furosemide and vaptan. These drugs can be used in lower doses. In one embodiment, “lower doses” means from 0.075% to 0.15% added to food (weight/weight).

In one embodiment, the present invention involves adenine analogs 2-chloro-6-aminopurine and/or (6-(Dimethylamino) purine, which are more potent than adenine and increase water excretion by the kidney in a dose-dependent manner. Adenine increases water loss by the kidney by altering the expression of the major water-absorbing channel in the kidney. The analogs of the present invention have an adenine nucleobase, which is a natural substance and is present in our cells as a constituent of DNA, RNA and energy molecules such as ATP and ADP. Hence, lower doses of adenine analogs or derivatives can be metabolized in the liver and cleared by the kidneys without causing any toxic effects.

As discussed above, adenine, a natural nucleobase molecule, impairs renal function, food behavior, and fluid balance in rats. Adenine at high dose causes a significant fluid loss by decreasing the gene expression and protein abundance of both water channel aquaporin 2 (AQP2) in the collecting duct system and the salt transporter NKCC2 in the medullary thick ascending limb of rat kidney. The present invention has additionally identified adenine analogs with modified molecular structure, which can mimic the effects of adenine on renal handling of salt and water in rat.

As shown in the examples below, 2-chloro-6-aminopurine and (6-(Dimethylamino) purine impair water balance and decrease urine osmolality in rat in a dose- and time-dependent manner. These effects are independent of changes in food behavior. The analogs R2-AND and R4-ADN are more potent than adenine in increasing water loss by the kidney.

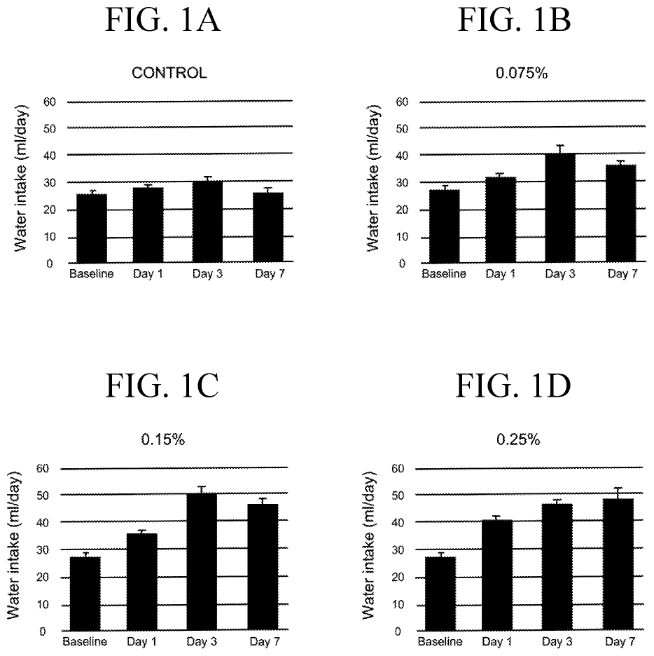

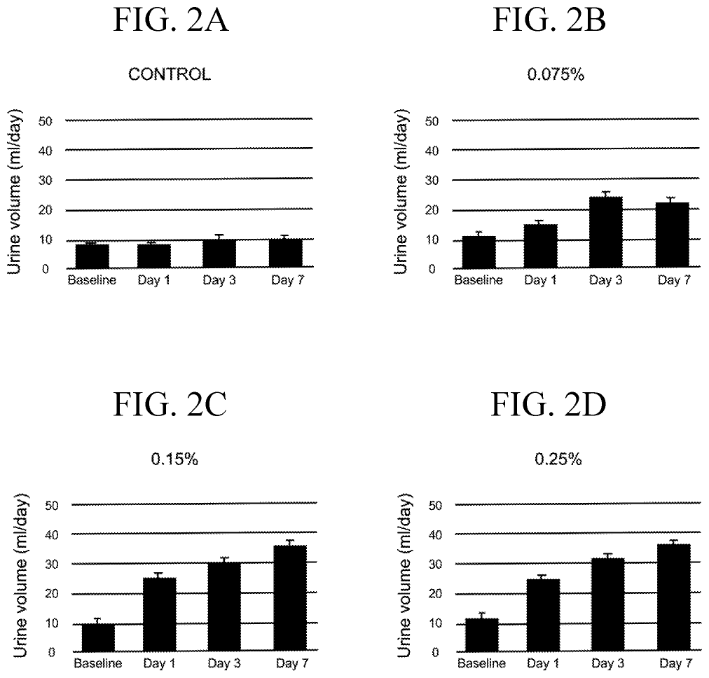

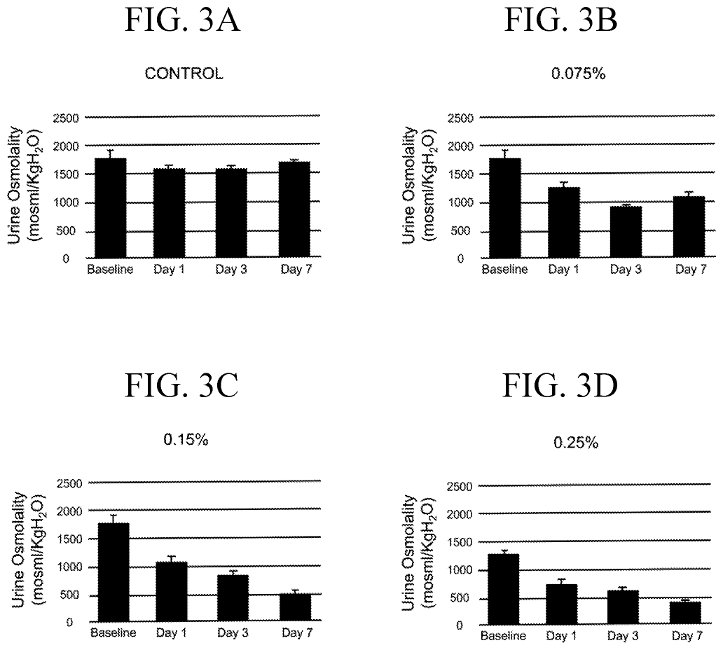

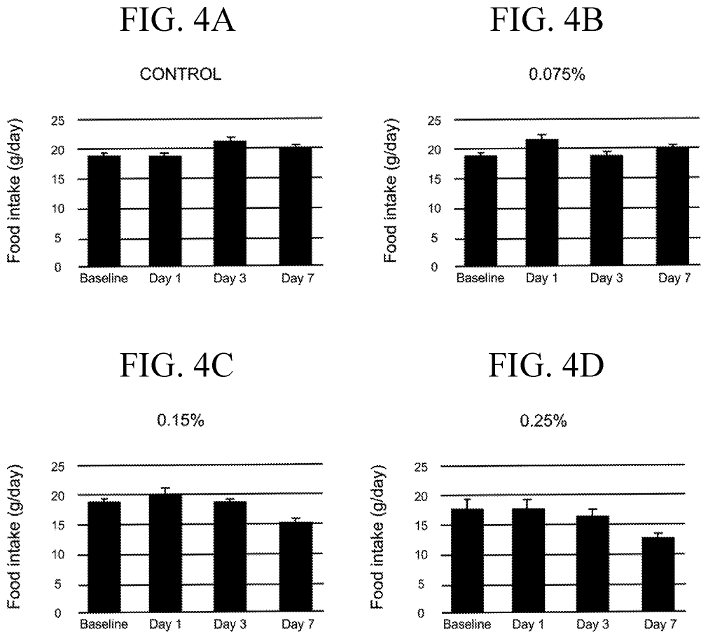

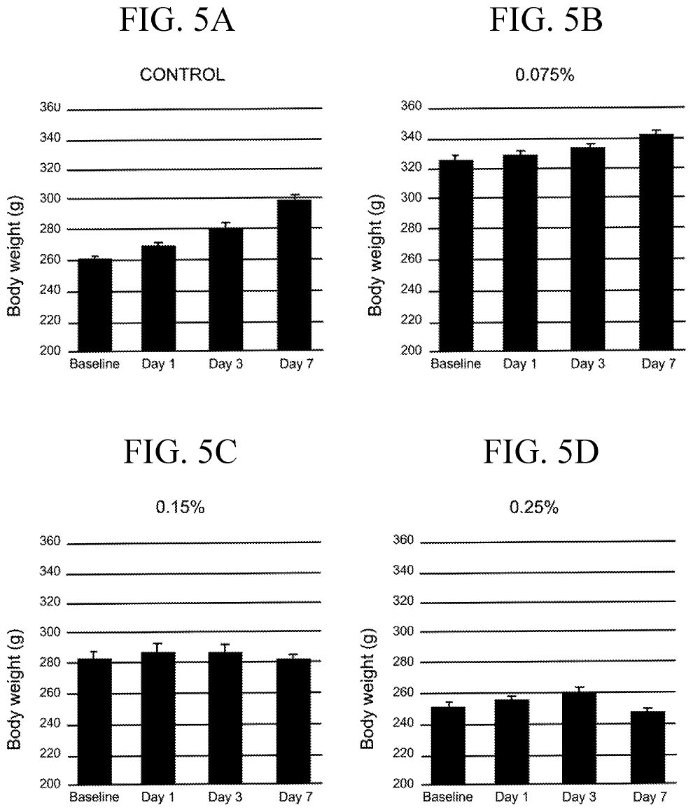

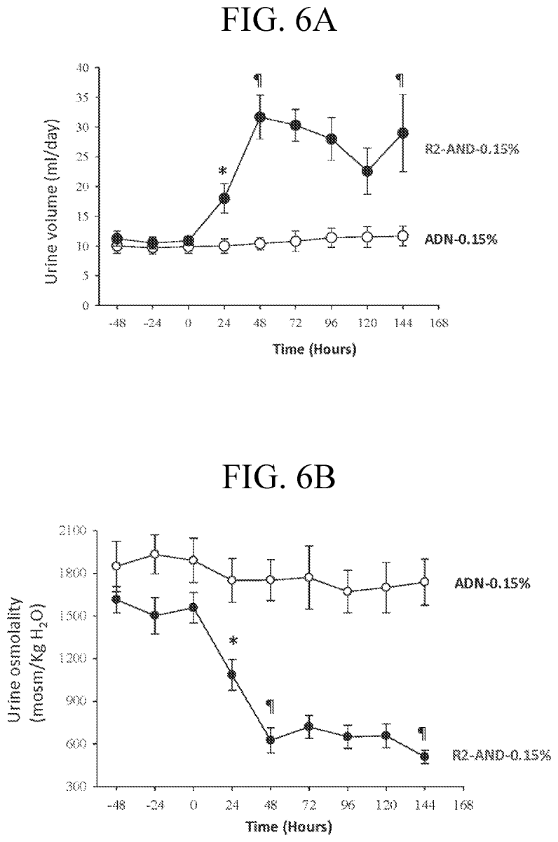

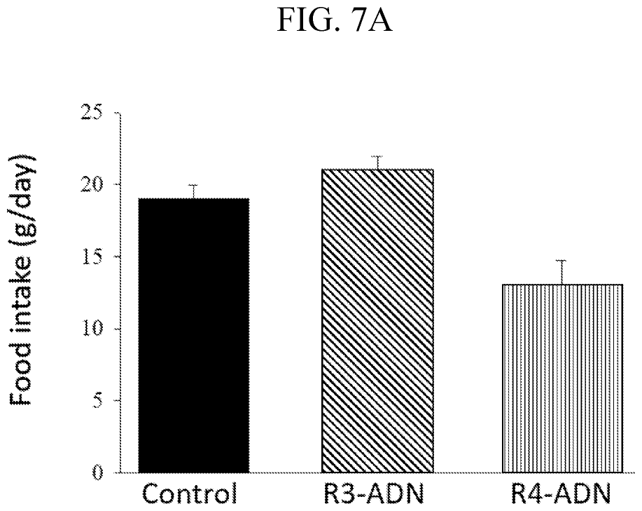

The results of Example 1 demonstrate that rats treated with the adenine analog R2-ADN exhibited a significant impairment in water balance as shown by a significant increase in water intake (FIGS. 1A-1D) and urine volume (FIGS. 2A-2D), as compared to both baseline levels and control rats. The increase in urine volume correlates with a sharp reduction urine osmolality (FIGS. 3A-3D) in R2-ADN-treated rats, as compared to both baseline levels or control rats. The effects of R2-ADN on these physiologic parameters are dose- and time-dependent. As shown in FIGS. 4A-4D, R2-ADN did not significantly alter food intake at lower doses, but a significant reduction in food intake was observed in rats treated with high dose (0.25%) on day 7 of R2-ADN feeding (FIGS. 4A-4D). These data clearly indicate that the impaired water balance in R2-ADN-fed rats is not linked to changes in food intake and rather resulted from renal effects of R2-ADN on water transport in the kidney. Lastly, it appears that the renal loss of fluid has impacted the changes in body weight of rats treated with R2-ADN, as compared to rats in the control group (FIGS. 5A-5D).

In addition, the effects of adenine (ADN) were compared to those of R2-ADN on these physiologic parameters. Urine osmolality data depicted in Table 1 show that rats fed R2-ADN at 0.15% exhibited a significant reduction (˜60%) in urine osmolality within the second day of treatment, as compared to even higher doses of adenine and throughout the entire period of treatment (Table 1). Further, while adenine at 0.15% has no effect on urine volume or urine osmolality, the same dose of R2-ADN significantly impaired daily water balance (FIG. 3A) and urine osmolality (FIG. 3B). Taken together, these data indicate that the analog R2-ADN is more potent than adenine in its effects of water handling by the kidney.

TABLE 1
Urine osmolality (mosm/kg H₂O) of untreated rats (Control) or rats treated with
differentdoses of adenine (ADN) or 0.15% R2-ADN. N = 4 to 5 rats in each group.

| | Baseline | Baseline | Average | | | | | | |
|---|---|---|---|---|---|---|---|---|---|
| | Day 1 | Day 2 | Baseline | Day 1 | Day 2 | Day 3 | Day 4 | Day 5 | Day 6 |
| Control | 1593 ± | 1457 ± | 1525 ± | 1717 ± | 1462 ± | 1831 ± | 1840 ± | 1833 ± | 1895 ± |
| | 134 | 97 | 109 | 165 | 115 | 114 | 117 | 93 | 116 |
| ADN- | 1848 ± | 1933 ± | 1890 ± | 1750 ± | 1752 ± | 1770 ± | 1671 ± | 1700 ± | 1738 ± |
| 0.15% | 178 | 137 | 157 | 154 | 144 | 221 | 150 | 177 | 163 |
| R2- | 1614 ± | 1502 ± | 1558 ± | 1085 ± | 625 ± | 720 ± | 649 ± | 657 ± | 508 ± |
| ADN- | 93 | 128 | 107 | 109 | 89 | 81 | 82 | 83 | 46 |
| 0.15% | | | | | | | | | |
| ADN- | 1638 ± | 1599 ± | 1619 ± | 1475 ± | 1371 ± | 1354 ± | 1296 ± | 1279 ± | 1386 ± |
| 0.20% | 65 | 42 | 48 | 114 | 120 | 94 | 66 | 44 | 99 |
| ADN- | 1648 ± | 1410 ± | 1529 ± | 1198 ± | 848 ± | 976 ± | 1019 ± | 938 ± | 956 ± |
| 0.25% | 108 | 136 | 120 | 112 | 79 | 54 | 69 | 46 | 72 |

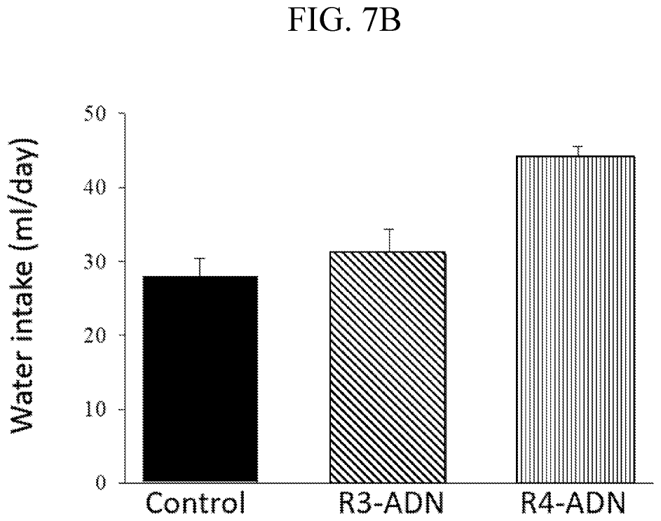

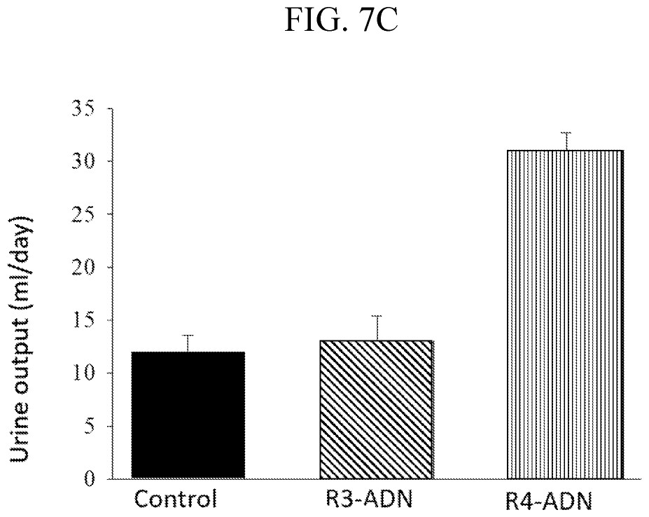

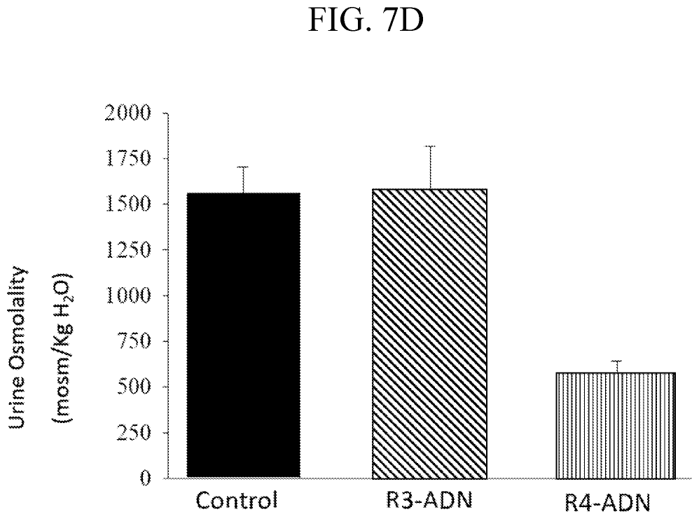

Regarding Example 2, the results demonstrate that rats treated with adenine analog R4-ADN [(6-(Dimethylamino) purine] at 0.15% (weight/weight) for 4 days exhibited a significant reduction in food intake (FIG. 7A) and significant impairment in water balance as shown by a significant increase in water intake (FIG. 7B) and urine volume (FIG. 7C), as compared to control rats. The increase in urine volume correlates with a sharp reduction urine osmolality (FIG. 7D) in R4-ADN-fed rats, as compared to control rats. Note that the effects on these parameters were developed as early as the day of R4-AND feeding (data not shown). Interestingly, none of the measured parameters (i.e. food intake, water intake, urine volume, and urine osmolality) were affected by R3-AND (N6-benzoyladenine) as compared to the Control groups (FIGS. 7A-D). These data demonstrate that 6-(Dimethylamino) purine at this lower dose (0.15%) significantly inhibits water reabsorption in the kidney and has the potential of being used as a therapeutic for the treatment of both hyponatremia and polycystic kidney disease.

It is well accepted in the medical community that the management of hyponatremia and the treatment of polycystic kidney disease represent a major healthcare burden in the U.S. Hence, safe and affordable new therapeutic agents like the ones disclosed in the present invention will significantly improve the management of hyponatremia and PKD and sharply reduce the healthcare cost associated with the treatments of these diseases.

### EXAMPLES

### Example 1

Male Sprague Dawley rats were placed in metabolic cages and fed powdered rodent chow with access to distilled water ad libitum. After adjustment, baseline data were collected for 2 days and then rats were randomly divided into different groups. Some rats remained on the same diet (Control) and some were switched to a powdered diet containing different doses (0.075%, 0.15% or 0.25%; weight/weight) of an adenine analog called 2-chloroadenine or 2-chloro-6-aminopurine (R2-ADN) and data were collected daily for 7 days.

### Example 2

Rats were placed in metabolic cages and fed control powdered diet or powdered diets supplemented with N6-benzoyladenine (R3-AND) or (6-(Dimethylamino) purine (R4-AND). All rats had free access to water. Food intake (FIG. 7A), water intake (FIG. 7B), urine volume (FIG. 7C) and urine osmolality (FIG. 7D), were measured at day 4 as compared to control. N=3 rats in each group.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method for treating hyponatremia in a mammal, the method comprising administering to the mammal in need of such treatment a therapeutically effective amount of an adenine analog selected from the group consisting of:
a. 2-chloro-6-aminopurine, its tautomer, racemate, optical isomer and/or pharmaceutically or nutritionally acceptable salt thereof, and a pharmaceutical carrier;
b. 6-(dimethylamino) purine, its tautomer, racemate, optical isomer and/or pharmaceutically or nutritionally acceptable salt thereof, and a pharmaceutical carrier;
c. and combinations thereof.

2. The method of claim 1 wherein the mammal is administered 2-chloro-6-aminopurine, its tautomer, racemate, optical isomer and/or pharmaceutically or nutritionally acceptable salt thereof, and a pharmaceutical carrier.

3. The method of claim 1 wherein the mammal is administered 6-(dimethylamino) purine, its tautomer, racemate, optical isomer and/or pharmaceutically or nutritionally acceptable salt thereof, and a pharmaceutical carrier.

4. The method of claim 1 wherein the mammal is administered a dosage of adenine analog from about 0.075% in food (weight/weight) to about 0.25% in food (weight/weight).

5. The method of claim 1 further comprising administering to the mammal one or more additional drugs to treat hyponatremia.

6. The method of claim 5 wherein the additional drugs are selected from the group consisting of furosemide, vaptans and combinations thereof.

7. A method for treating polycystic kidney disease in a mammal, the method comprising administering to the mammal in need of such treatment a therapeutically effective amount of an adenine analog selected from the group consisting of:
a. 2-chloro-6-aminopurine, its tautomer, racemate, optical isomer and/or pharmaceutically or nutritionally acceptable salt thereof, and a pharmaceutical carrier;
b. 6-(dimethylamino) purine, its tautomer, racemate, optical isomer and/or pharmaceutically or nutritionally acceptable salt thereof, and a pharmaceutical carrier;
c. and combinations thereof.

8. The method of claim 7 wherein the mammal is administered 2-chloro-6-aminopurine, its tautomer, racemate, optical isomer and/or pharmaceutically or nutritionally acceptable salt thereof, and a pharmaceutical carrier.

9. The method of claim 7 wherein the mammal is administered 6-(dimethylamino) purine, its tautomer, racemate, optical isomer and/or pharmaceutically or nutritionally acceptable salt thereof, and a pharmaceutical carrier.

10. The method of claim 7 wherein the mammal is administered a dosage of adenine analog from about 0.075% in food (weight/weight) to about 0.25% in food (weight/weight).

11. The method of claim 7 further comprising administering to the mammal one or more additional drugs to inhibit cyst fluid filling and cyst growth in polycystic kidney disease.

12. The method of claim 7 wherein the adenine analog is administered orally.

13. The method of claim 7 wherein the adenine analog is administered via subcutaneous injection.

14. The method of claim 7 wherein the adenine analog is administered intravenously.
